(19) United States
(12) Patent Application Publication
(10) Pub. No.: US 2005/0048021 A1
(43) Pub. Date: Mar. 3, 2005

(54) Proportional method for diagnosing and appropriately cleansing and conditioning hair and a kit of proportional shampoos and conditioners for practicing the method

(76) Inventors: Robert Salem, New York, NY (US); Doina Sandulache, Yonkers, NY (US); Maria Iliopoulos Laguardia, New York, NY (US)

Correspondence Address: John F. Gulbin;Pitney, Hardin, Kipp & Szuch LLP, 685 Third Avenue, New York, NY, 10017-4024, US

(21) Appl. No.: 10/650,356

(22) Filed: Aug. 27, 2003

(51) Int. Cl. A61K 7/06; A61K 7/11; G06F 17/60

(52) U.S. Cl. 424/70.14; 705/2

(57) ABSTRACT

A method for diagnosing and appropriately cleansing and conditioning hair includes steps of evaluating characteristics of the hair and scalp and cleansing and conditioning the hair with a shampoo and a conditioner selected from a kit having a plurality of shampoos and conditioners based upon the results of the evaluations. The shampoos and conditioners in the kit include graduated amounts of surfactants and conditioning polymers, so that a shampoo and a conditioner most suitable for a specific hair and scalp may be selected and used.

### BACKGROUND OF THE INVENTION

1. Field of the Invention

The present invention relates to the hair care field. More particularly, it relates to the cleansing and conditioning of hair with shampoos and conditioners intended to address specific characteristics of the hair and scalp and to achieve specific desired results.

2. Description of the Prior Art

Shampoos and conditioners are well known to those of ordinary skill in the hair-care art, as well as to consumers who have a wide variety of them from which to choose at the retail level. In general, shampoos and conditioners fall into two broad categories: cosmetic shampoos and conditioners and medicated shampoos and conditioners. Use of the former is primarily driven by consumer expectation for a specific desired result, while use of the latter is based on a medical condition of the scalp. In either case, one objective of shampooing is to clean natural oils, environmental soils and styling product residue from the hair, and one objective of conditioning is to detangle, reduce or remove static, impart shine and improve wet and dry combing.

In addition to these objectives, shampoos and conditioners have been aligned with one another in certain instances to address the needs of hair having some particular characteristic. For example, shampoos and conditioners are paired and widely available for oily, normal and dry hair, these being categories of hair type.

The present invention is primarily concerned with cosmetic shampoos and conditioners, and the remarks made in the preceding paragraphs may be generalized for them as follows: shampoos and conditioners perform separate functions and are grouped together by manufacturers to deliver coordinated end benefits. As such, shampoos and conditioners are generally sold as companions, which are codependent on each other, and which most often perform against a single hair characteristic, specified in terms of an indication on the product label.

Those who regularly shop for shampoos and conditioners in retail stores, or who visit hair or beauty salons, will recognize that they are sold under many such indications, including not only the oily, normal and dry mentioned above, but also damaged, weak, permed, fragile and others. Generally speaking, shampoos and conditioners are organized to address six principal cosmetic “segments” by manufacturers. For example, the “hair type” segment includes the indications “oily”, “normal” and “dry”.

The other segments are “Hair Condition”, “Hair Texture”, “Tensile Strength”, “Prior Use of Chemical Processes” and “Beauty End Benefit”, each of which has two or more indications, that is, hair characteristics that may appear on the labels of products intended to address them. These will be summarized for the six segments in Table 1 below.

TABLE 1

| Segment | Indication |
|---|---|
| Hair Type | Oily, Normal, Dry |
| Hair Condition | Damaged, Dry Ends |
| Texture | Fine, Normal, Coarse |
| Tensile Strength | Weak, Fragile |
| Chemical Process | Color-Treated, Permed |
| Beauty End Benefit | Volume, Curl, |
| | Straight |

Those familiar with shampoos and conditioners will recognize that most brands currently available on the market generally address only a single segment. That is, a given manufacturer's shampoo/conditioner paired products may only be available in “oily”, “normal” and “dry”. Occasionally, two segments, at most, are addressed in a single product.

In reality, a given consumer's hair has several needs which arise independently from one another for different reasons. For example, hair type, texture and curvature and the scalp's rate of sebum (oil) production are genetically determined. Chemical and mechanical processes to which the hair has previously been subjected may temporarily affect its condition. Environmental conditions, such as weather and seasonal weather patterns, also have their effect on hair, as do the styling products used on it. As a consequence, a given consumer's hair may be amenable to characterization under several of the so-called indications, making it virtually impossible to determine which shampoo/conditioner pair would be most suitable.

A couple of examples will illustrate this problem. Imagine that a consumer has a dry scalp and highlighted and color-treated coarse, wiry hair that is also damaged. She desires mild cleansing that will prevent the flaking that can accompany dry scalp. In addition, she wants repair and protection for her damaged hair. Should she buy a shampoo and a conditioner that improve her scalp dryness, or for color-treated hair, or for repair and protection for her damaged hair, or to create a sleek effect? One can see that she has at least four possible shampoo and conditioner systems to choose from.

Let's consider another example. A consumer has an oily scalp that she needs to cleanse daily. In addition, she has fine hair that is weak and color-treated. Among the possibilities for shampoo and conditioner are a system for oily scalp, one for volume to enhance fine texture, one to add strength, one for color-treated hair, one to enhance volume, and one to improve strength as fine hair tends to lack tensile strength.

In reality, none of the above shampoo and conditioner systems is truly ideal for these consumers' hair.

In view of these two examples, it is clear that the consumers would have to buy seven or eight individual products to address all of their shampoo and conditioning needs. Of course, they could not use all of them each time they cleansed and conditioned their hair. Instead, they would tend to alternate which products they use and, as a result, would not receive benefits that meet all of their hair needs each time they shampoo and condition. Moreover, if they did try to use four shampoos and four conditioners at the same time, they could not expect optimal results as the shampoos and conditioners might not be compatible with one another.

Because of these shortcomings of the shampoos and conditioners of the prior art, it is an object of the present invention to broaden the range of segments and indications taken into consideration when formulating shampoos and conditioners.

More specifically, it is an object of the present invention to address individual scalp needs, such as rate of oil or sebum production, such as oily, normal/oily, normal/dry or dry; texture, whether fine, medium, coarse or very coarse; hair condition, such as chemically treated; hair shape, for example, straight or curly; hair's tensile strength, such as weak and in need of reinforcement; and damage that may occur due to chemical, mechanical or environmental factors in specific shampoo and conditioner formulas.

### SUMMARY OF THE INVENTION

Accordingly, the present invention is a method for diagnosing and appropriately cleansing and conditioning hair. In its broadest form, the method comprises the steps of evaluating a scalp to determine the rate of sebum production thereof, and of evaluating the hair to determine the texture thereof. Based on the results of these evaluations, the next step of the method is to recommend a proportional shampoo from a plurality of proportional shampoos, wherein individual proportional shampoos in said plurality include anionic, nonionic and amphoteric surfactants and cationic conditioning polymers, in graduated amounts relative to one another to provide incremental cleansing and conditioning for specific scalp types and hair textures.

The next step of the method is to cleanse the hair and scalp with the recommended proportional shampoo. Following this cleansing step, the method continues with the steps of verifying the texture determined earlier, and of evaluating the state of the hair. Based on the results of these evaluations, the next step in the practice of the method is to recommend a proportional conditioner from a plurality of proportional conditioners, wherein individual proportional conditioners in said plurality include at least one amphoteric surfactant, at least one cationic conditioning polymer, at least one film former, and at least one zwitterionic compound selected from the group consisting of amino acids, proteins and combination thereof, in graduated amounts relative to one another to provide incremental conditioning, repair, strengthening, shine and protection for specific textures and hair states.

The final step of the method is to condition the hair with the recommended proportional conditioner.

The present invention is also a kit of proportional shampoos and conditioners to enable one to practice the method. The kit comprises a plurality of proportional shampoos, wherein individual proportional shampoos in said plurality include anionic, nonionic and amphoteric surfactants and cationic conditioning polymers, in graduated amounts relative to one another to provide incremental cleansing and conditioning for specific scalp types and hair textures.

The kit also comprises a plurality of proportional conditioners, wherein individual proportional conditioners in said plurality include at least one amphoteric surfactant, at least one cationic conditioning polymer, at least one film former, and at least one zwitterionic compound selected from the group consisting of amino acids, proteins and combination thereof, in graduated amounts relative to one another to provide incremental conditioning, repair, strengthening, shine and protection for specific textures and hair conditions.

The kit finally comprises a diagnostic means to identify the appropriate proportional shampoo from said plurality of proportional shampoos and the appropriate proportional conditioner from said plurality of proportional conditioners for the scalp and hair of a particular person. The diagnostics means may, for example, be a set of written instructions for use by a hair-care professional and one or more flow charts for use with the instructions to choose the appropriate shampoo and conditioner.

The present inventions will now be described in more complete detail, with reference being made in the discussion to follow to the figures identified below.

### BRIEF DESCRIPTION OF THE DRAWINGS

FIG. 1 is a diagram illustrating the relationship of the proportional shampoos and conditioners of the present invention to five (5) distinct criteria for hair assessment.

FIG. 2 is the diagram of FIG. 1 further illustrating the relationship of component charges with the proportional shampoos and conditioners of the present invention.

FIG. 3 is a bar chart illustrating the relative weight percent of each category of active components contained in the proportional shampoos.

FIG. 4 is a bar chart illustrating the relative weight percent of each category of active components contained in the proportional conditioners.

FIG. 5 is a flow chart illustrating the determination of the proportional shampoo to be used for the eight combinations of scalp type and hair texture.

FIG. 6 is a flow chart illustrating the determination of the proportional conditioner to be used for the eight combinations of hair texture and condition of hair.

FIG. 7 is a bar chart illustrating color intensity of swatches of bleached and normal hair after ten treatments of shampooing and conditioning.

FIG. 8 is a bar chart illustrating hair characteristics, as determined by a panel, of tresses of single-processed bleached hair after ten treatments of shampooing and conditioning.

FIG. 9 is a pie chart illustrating the overall satisfaction by participants in a home-use-study with shampoos selected using the method of the invention as compared to shampoos most often used by the participants.

FIG. 10 is a pie chart illustrating the overall satisfaction by participants in a home-use-study with conditioners selected using the method of the invention as compared to conditioners most often used by the participants.

FIG. 11 is a pie chart illustrating the overall combined satisfaction by participants in a home-use study with the shampoos and conditioners selected using the method of the invention as compared to the shampoos and conditioners most often used by the participants.

### DETAILED DESCRIPTION OF THE PREFERRED EMBODIMENTS

Before discussing the present invention in detail, its rationale will be briefly presented and some definitions of terms used in the description and practice of the invention will be given. Following the definitions, the rationale will be given in more complete detail.

### I. Basic Rationale

In accordance with the present invention, both scalp type and hair texture are used to determine the shampoo needs of an individual consumer. The scalp supplies sebum to the hair. Sebum keeps the surface of the hair and scalp smooth and supple, and protects the hair from excessive loss of emolliency. Sebum production is genetically determined and controlled hormonally. Accordingly, the rate of sebum production varies from individual to individual. Four generally recognized categories are used to describe the level of sebum production on the scalp: Oily, Normal/Oily, Normal/Dry and Dry, although, it should be understood, the range in the level of sebum production observed in individuals may be broken down into more narrowly defined categories than these four, or fewer broadly defined categories.

Hair texture is determined by its diameter, expressed as the degree of fineness and coarseness of each individual strand of hair, as well as its smoothness and roughness. Texture is important because it determines how easily sebum is transported from the scalp along a hair strand towards its mid-shaft. Textures are generally expressed in terms of the following four categories: Fine, Medium, Coarse and Very Coarse, although textures may be broken down into more narrowly defined categories than these four, or fewer broadly defined categories.

As stated, both scalp and texture are important when making a shampoo selection. For example, an oily scalp requires more cleansing than a dry scalp. If the hair itself is of a fine texture, it is more likely to accumulate sebum because of its diameter and the resulting ease with which sebum can be transported therealong from the scalp. Accordingly, if the scalp is oily and the hair texture is fine, sebum will travel along the hair from the scalp easily, lubricating and protecting it. However, this particular combination of scalp and texture also results in limpness, hair cohesiveness and quick accumulation of dirt. To select a shampoo for these two indications (oily scalp and fine texture) is relatively simple—one needs a high-cleansing shampoo that will not weigh down the hair.

When the indications are more extreme, for example, with oily scalp and coarse-textured hair, they present very separate considerations. While the scalp still requires more cleansing, coarse hair is relatively unaffected by the sebum which is not readily transported therealong from the scalp because it is rougher and thicker than fine hair. Therefore, milder cleansing and some emollients are needed to moisturize and soften coarse hair.

In accordance with the present invention, both hair texture and the state of the hair are used to determine the conditioning needs of an individual consumer. The existing state of the hair, namely, its porosity, elasticity and strength, level of emolliency and evidence of previous treatments, such as color treatment and presence of damage. In general, the state of hair is determined by genetic disposition, and affected by mechanical and environmental factors and chemical alteration.

While hair of all four textures has the same basic conditioning needs—static reduction and wet and dry combing improvement—the current state of the hair of each texture type requires different levels of repair, strengthening, protection and shine.

For example, fine hair with a double-process hair color requires the same basic conditioning as coarse hair with a double-process hair color. However, due to its texture, fine hair has more strengthening needs than coarse hair.

Having presented this basic rationale, some definitions will now be given for terms used in the discussion as they apply to the present invention.

### II. Definitions

We first turn to definitions relating to scalp function. As noted above, the scalp supplies lipids (sebum) to the hair. Sebum is produced continuously by the sebaceous glands and acts as a lubricant, keeping the surface of the hair and scalp smooth and supple, and forms a slightly oily protective coating over the hair to help prevent excessive loss of emolliency. Sebum production is genetically determined and controlled hormonally. Accordingly, the rate of sebum production varies from one individual to the next, and may vary with time for a given individual. The four generally recognized categories that describe the level of sebum production on the scalp are characterized in Table 2 below.

TABLE 2

| Scalp Type | Characteristics |
|---|---|
| Oily | Excessive sebum production; |
| | Scalp and hair roots look oily; |
| | Depending on the texture of the hair, causes |
| | varying degrees of limpness and |
| | cohesiveness; |
| | The sebum attracts dirt and the hair appears |
| | and feels dirty quickly; |
| | Re-oiling occurs rapidly, requiring washing |
| | within 24 hours. |
| Normal/Oily | Normal sebum production with a tendency |
| | towards oiliness; |
| | Scalp is normal and healthy; |
| | Hair appears shiny from the sufficient |
| | amount of sebum; |
| | Dirt and sebum will accumulate, requiring |
| | washing within 48 hours. |
| Normal/Dry | Normal sebum production with a tendency |
| | towards dryness; |
| | Scalp is normal and healthy but sometimes |
| | feels dry; |
| | Hair begins to appear shiny when re-oiling |
| | begins to occur within about 48 hours |
| | after washing; |
| | Dirt begins to dull the hair 72 hours after |
| | washing. |
| Dry | Insufficient sebaceous secretion; |
| | Scalp appears taut and dry, and flaking may |
| | occur; |
| | Little or no sebum is transferred to the |
| | hair, causing it to become dull-looking |
| | and brittle; |
| | Due to lack of sebum, rarely needs to be |
| | washed, but scalp may begin to flake 96 |
| | hours after washing. |

Another method of identifying the scalp type of a particular individual is the time period between required washings. An approximate correlation between scalp type and washing intervals is summarized in Table 3.

TABLE 3

| | Time Washing Required |
|---|---|
| Scalp Type | (since previous washing) |
| Oily | 24 Hours |
| Normal/Oily | 48 Hours |
| Normal/Dry | 72 Hours |
| Dry | 96 Hours |

We now consider the various textures by which hair is generally classified. As noted above, hair texture is the degree of fineness or coarseness, that is, thinness or thickness, of each strand of hair, encompassing the diameter of the hair, as well as its smoothness and roughness. Fineness and coarseness are genetically determined. The smoothness and roughness of hair are impacted by genetics, mechanical considerations, chemical processes and environmental effects. Texture determines how easily sebum is transported from the scalp toward the mid-shaft of the hair, whether on its own or by mechanical means, such as, brushing or combing.

The four categories generally used to describe hair texture are characterized in Table 4 below.

TABLE 4

| Hair Texture | Characteristics |
|---|---|
| Fine | Hair diameter is below average; |
| | Lacks firmness and rigidity; |
| | Subject to flyaway and tangles easily; |
| | Weak and sensitive to external trauma, |
| | splitting and breaking easily; |
| | Due to lack of tensile strength, tends to |
| | have difficulty holding a style. |
| Medium | Hair diameter is average; |
| | Hair is both firm and soft; |
| | Easy to detangle both when wet and when dry; |
| | Due to good tensile strength, holds a style |
| | very well. |
| Coarse | Hair diameter is larger than average; |
| | Tends to appear rougher than medium hair, |
| | but is less susceptible to external and |
| | mechanical traumas; |
| | Tends to be unruly and tangles easily. |
| Very Coarse | Hair diameter is much larger than average; |
| | Extremely rough to the touch and difficult |
| | to manage; |
| | Resists combing, brushing and styling |
| | efforts. |

The following are definitions of the terms used to describe the current state of the hair. As noted above, the state of the hair, namely, its porosity, elasticity, strength, level of emolliency and evidence of previous treatments and presence of damage. State of the hair is determined by genetic disposition, such as curly or straight; by mechanical factors, including brushing, combing and styling; and environmental factors, including sun, pollution and water. Chemical alteration, including color, bleaching, and permanent waving, also have effect on the state of the hair. Characterizations for the state of the hair are summarized in Table 5 below.

TABLE 5

| State of the Hair | Characterization |
|---|---|
| Normal | Good tensile strength, smooth cuticle and |
| | natural shiny appearance; |
| | Virgin to chemical alteration; |
| | Not significantly affected by mechanical |
| | or environmental implications. |
| Single-Processed | Chemically altered by the application of |
| | one color product, lifting no more than |
| | four levels, either all over or |
| | partially distributed by highlighting. |
| Double-Processed | Colored in with two separate procedures in |
| | which the hair is first decolorized or |
| | pre-lightened with a lightener before |
| | the depositing color is applied; |
| | Hair can become weakened and have |
| | diminished tensile strength, porosity |
| | and elasticity. |
| Naturally Curly | Genetically determined, grows in varying |
| | degrees of long twisted spirals; |
| | Shows varying diameters along each strand; |
| | Thinnest and weakest sections of the hair |
| | strand are located at the twists. |
| Permanent-Waved | Impacted by both a mechanical action and a |
| or Straightened | chemical action in which the bonds of |
| | the hair are broken and reformed in a |
| | new wavy, curly or straight state; |
| | Hair can become weakened and have |
| | diminished tensile strength, porosity |
| | and elasticity. |
| Damaged | Environmentally, mechanically and |
| | chemically altered; |
| | Loss of sheen, brittle to the touch, |
| | abraded cuticle, over-porous, and |
| | breaks easily. |

### III. Scalp/Texture Relationships For Cleansing

Having these definitions at hand, we now summarize more completely the relationship between scalp and texture as they relate to cleansing or shampooing. The standard for any comparisons is virgin, medium-textured hair. However, due to the combinations of different scalp types and hair texture, at least eight (8) different possible combinations with varying characteristics are to be considered.

A) Oily Scalp—Fine and Medium Hair

| | |
|---|---|
| Diameter | Sebum is transported down the hair |
| | easily due to the smooth narrow to |
| | medium surfaces. |
| Appearance | Hair clumps easily and can appear |
| | oily, limp and dull. |
| Environment | Hair is protected from environmental |
| | damage because of sebum. |
| Soils | Dirt accumulates quicker due to the |
| | affinity of dirt to the sebum. |

B) Oily Scalp—Coarse and Very Coarse Hair

| | |
|---|---|
| Diameter | Hair diameter is larger than average |
| | causing it to resist the transport |
| | of oil from root to mid-shaft. |
| Appearance | Due to the texture, there is no |
| | limpness from the sebum but hair |
| | appears dull and rough to the |
| | touch due to lack thereof. |
| Environment | Hair is exposed to environmental |
| | damage due to lack of sebum. |
| Soils | Scalp feels soiled quickly but the |
| | hair is relatively unaffected by |
| | the sebum. Each of these |
| | conditions is a separate |
| | consideration. |

C) Normal/Oily Scalp—Fine and Medium Hair

| | |
|---|---|
| Diameter | Sebum is transported down the hair |
| | easily due to the hair's diameter |
| | and smooth surface. |
| Appearance | Hair appears shiny from the |
| | sufficient amount of sebum. |
| Environment | Hair is protected from environmental |
| | damage because of sebum. |
| Soils | Hair and scalp will begin to appear |
| | soiled 48 hours after washing. |

D) Normal/Oily Scalp—Coarse and Very Coarse Hair

| | |
|---|---|
| Diameter | Scalp is healthy but, although it |
| | transfers sebum to the hair at the |
| | root level, the hair resists |
| | transporting the sebum to mid- |
| | shaft due to its larger diameter. |
| Appearance | Hair appears dull and rough to the |
| | touch due to lack of sebum. |
| Environment | Hair is exposed to environmental |
| | damage due to lack of sebum. |
| Soils | While the scalp secretes a normal |
| | amount of oil, the hair is |
| | relatively unaffected by the |
| | sebum. Each of these conditions is |
| | a separate consideration. |

E) Normal/Dry Scalp—Fine and Medium Hair

| | |
|---|---|
| Diameter | Sebum is transported down the hair |
| | easily due to the hair's diameter |
| | and smooth surface, but scalp has |
| | a tendency towards dryness. |
| Appearance | Hair appears shiny and healthy when |
| | re-oiling begins to occur, |
| | approximately 48 hours after |
| | washing. |
| Environment | Hair is exposed to environmental |
| | damage due to low sebum. |
| Soils | Scalp and hair will appear soiled 72 |
| | hours after washing. |

F) Normal/Dry Scalp—Coarse and Very Coarse Hair

| | |
|---|---|
| Diameter | Scalp is healthy with a slight |
| | tendency to be dry and, although |
| | re-oiling of the scalp begins to |
| | occur 48 hours after washing, the |
| | larger diameter of the hair |
| | resists the transport of sebum. |
| Appearance | Hair is dull and rough to the touch. |
| | Hair fibers are separated due to |
| | lack of emolliency. |
| Environment | Hair exposed to environmental damage |
| | due to low sebum. |
| Soils | Although the scalp will appear soiled |
| | 72 hours after washing, the hair |
| | is unaffected by the sebum. |

G) Dry Scalp—Fine and Medium Hair

| | |
|---|---|
| Diameter | Scalp is taut, dry and tends to |
| | flake. Hair is dry, dull and |
| | fragile due to its narrow diameter |
| | and lack of sebum. |
| Appearance | Hair tangles and ends split easily. |
| Environment | Hair is exposed to environmental |
| | damage because of lack of sebum. |
| Soils | Scalp flaking from dryness 96 hours |
| | after washing drives the frequency |
| | of washing. |

H) Dry Scalp—Coarse and Very Coarse Hair

| | |
|---|---|
| Diameter | Scalp is taut, dry and tends to |
| | flake. Hair is dry and brittle due |
| | to lack of sebum. |
| Appearance | Hair is dull, and hard to manage. |
| Environment | Hair is exposed to environmental |
| | damage because of lack of sebum. |
| Soils | Scalp flaking from dryness 96 hours |
| | after washing drives the frequency |
| | of washing. |

### IV. Texture/Condition Relationships for Conditioning

The relationship between textures and conditions of hair as they relate to conditioning are now summarized more completely.

A) Fine/Normal or Single-Processed

Where fine hair is normal or single-processed, it may lose a small amount of its tensile strength and elasticity, and therefore requires conditioning and strengthening.

B) Fine/Naturally Curly, Double-Processed or Permed

Where fine hair is naturally curly, or has gone through multiple color processes or is permed, it may lose a significant amount of tensile strength and elasticity, and requires moderate conditioning in addition to repair and protection.

C) Medium/Normal or Single-Processed

Where medium hair is normal or single-processed, it may lose a small amount of its tensile strength and elasticity, and requires a moderate level of conditioning in addition to repair and protection.

D) Medium/Naturally Curly, Double-Processed or Permed

Where medium hair is naturally curly, or has gone through multiple color processes or is permed, it may lose some of its tensile strength, porosity and elasticity, may be dry to the touch, and requires a high level of conditioning in addition to repair and protection.

E) Coarse/Normal or Single-Processed

Where coarse hair is normal or single-processed, it may lose a small amount of its elasticity and emolliency. Because of its rough surface, with or without any single-process treatment, this type of hair requires a high level of conditioning in addition to repair and protection.

F) Coarse/Naturally Curly, Double-Processed or Permed

Where coarse hair is naturally curly, or has gone through multiple color processes or is permed, it may loose some of its elasticity and emolliency. Because of its rough surface, with or without any chemical interaction, this type of hair requires a very high level of conditioning in addition to repair and protection.

G) Very Coarse/Normal or Single-Processed

Where very coarse hair is normal or single-processed, it may loose a small amount of its elasticity and emolliency. Because of its very rough, brittle natural state, this type of hair requires a very high level of conditioning in addition to repair and protection.

H) Very Coarse/Naturally Curly, Double-Processed or Permed

Where very coarse hair is naturally curly, or has gone through multiple color processes or is permed, it may lose some of its elasticity and emolliency. Because of its very rough, brittle natural state, this type of hair requires, in addition to basic conditioning, a higher level of repair, strengthening and emolliency.

It is important to add that where hair in any of these eight texture/condition categories has been damaged by mechanical, environmental or chemical means, it may require additional repair and protection, in addition to basic conditioning above, than indicated.

### V. Proportional Cleansing and Conditioning Method

With the preceding background in place, the present invention is, in part, a proportional cleansing and conditioning method, which encompasses three separate aspects, which are:
A) A diagnostic aspect that broadens the range of indications considered when formulating shampoos and conditioners;
B) A product aspect designed to align (i.e., correlate) the indications to the individual needs of the consumer through proportional cleansing and conditioning; and
C) A scientific aspect to enable the products to perform on the diagnosed substrates (that is, the hair).

Each of these three aspects will now be discussed in detail.

### A. The Diagnostic Aspect

The diagnostic aspect broadens the range of indications considered when formulating shampoos and conditioners. The diagnostic aspect describes the characteristics of the “substrates”, that is, the hair, and establishes the bases for providing an assortment of cleansing and conditioning products.

The diagnostic aspect has been developed through empirical testing and is rooted in the theory that an individual's hair and scalp can be diagnosed and subsequently aligned with cleansing and conditioning products containing proportional (i.e., graduated) levels of surfactants and conditioning ingredients. That is to say, more specifically, both the shampoos and conditioners with which the method of the invention is practiced contain graduated levels of cleansing and conditioning (which includes basic conditioning, repair, protection, strengthening and shine) activity.

In accordance with the method, the cleansing and conditioning needs of each individual are determined, and, from a plurality of shampoos and conditioners, an appropriate shampoo and conditioner that address the needs of the individual are selected. In accordance with the invention, this is accomplished by evaluating at least three (3) separate criteria for an individual through observation and responses to a set of predetermined questions: (1) the individuals scalp; (2) the texture of the individual's hair; and the condition of the individual's hair. The observations and responses are then compared to a set of predetermined possibilities. This systematic process of the invention is performed in 2 phases: the “Shampoo Diagnosis” and the “Conditioner Diagnosis”.

1) The Shampoo Diagnosis

In the Shampoo Diagnosis, the scalp is inspected and evaluated to assess the rate of sebum production and whether any flaking is present to determine the proper proportion of cleansing agents (that is, surfactants) needed in the shampoo to be used. The texture of the hair is also evaluated to determine whether the hair is fine, medium, coarse or very coarse, as the texture determines the level of protection (that is, conditioning) needed in the shampoo.

In accordance with the invention, the Shampoo Diagnosis proceeds in four (4) steps:
a) Step 1—Determination of Scalp Type: The scalp is inspected to look for excess oil (sebum) production and any flaking. An answer to the question “How often do you wash your hair?” is obtained. If the answer is once every two to four (or more) days, the scalp guidelines table (was previously set forth in Table 3, supra) gives the scalp condition. If the answer is daily, an answer to an additional question “If you did not shampoo daily, how long would it take after shampooing for your scalp and hair to begin to look soiled and in need of cleansing?” is obtained. The answer, which will be from daily to once every four (or more) days, is then used to find the scalp condition in the scalp guidelines, which was previously set forth in Table 3, supra.
b) Step 2—Determination of Hair Texture: The hair is inspected visually and by touch to determine its diameter, and felt to determine its degree of roughness or smoothness. An answer to the question “Does your hair tangle or break easily?” is obtained. The answer indicates whether the hair is rough, if it tangles, or smooth, if it does not, as well as its tensile strength, if it breaks easily or does not. An answer to an additional question “How well does your hair style and hold a style?” is then sought. The answer indicates whether the texture is coarse or very coarse, if it is difficult to style, or whether it is fine, if it does not hold a style.
c) Step 3—Shampoo Selection: The information obtained in Steps 1 and 2 is then used to make a preliminary determination of the requisite level of cleansing for the individual's hair and thus a basic shampoo recommendation in accordance with Table 6 below.

d) Step 4—Reevaluation of Shampoo Selection: The hair, if fine or medium, is further inspected to determine whether it is damaged. If it is, a shampoo with proportionally more conditioning activity may be recommended in accordance with Table 6 to compensate for the damage.

TABLE 6

| | Hair Texture | |
|---|---|---|
| | and/or | |
| Scalp Type | Damage | Cleansing needed |
| Oily | Fine/Medium | High cleansing shampoo |
| Oily | Fine/Medium | Moderately high cleansing |
| | (Damaged) | shampoo |
| | | Emollients added to moisturize |
| | | Conditioning ingredients to |
| | | protect |
| Oily | Coarse/ | Moderately high cleansing |
| | Very Coarse | shampoo |
| | | Emollients to moisturize |
| Normal/Oily | Fine/Medium | Moderately high cleansing |
| | | shampoo |
| Normal/Oily | Fine/Medium | Moderately high cleansing |
| | (Damaged) | shampoo |
| | | Emollients added to soften and |
| | | protect the hair |
| Normal/Oily | Coarse/ | Moderate level of cleansing |
| | Very Coarse | shampoo |
| | | Emollients added to soften |
| | | hair's texture |
| Normal/Dry | Fine/Medium | Moderate level of cleansing |
| Normal/Dry | Fine/Medium | Gentle cleansing |
| | (Damaged) | Emollients added for |
| | | protection |
| Normal/Dry | Coarse/ | Gentle cleansing |
| | Very Coarse | Emollients added to soften |
| | | hair's texture |
| | | Emollients added to protect |
| | | hairs |
| Dry | Fine/Medium | Gentle cleansing |
| | | Emollients added to protect |
| | | the hair |
| Dry | Fine/Medium | Gentle cleansing |
| | (Damaged) | Conditioning ingredients added |
| | | to protect the hair |
| Dry | Coarse/ | Gentle cleansing |
| | Very Coarse | Emollients added to soften |
| | | hair's texture |
| | | Conditioning ingredients added |
| | | to protect the hair |

2) The Conditioner Diagnosis

The texture of the hair, as determined in the Shampoo Diagnosis, has a direct bearing on the proportion of strengthening and conditioning needed in the conditioner to be used. The condition of the hair, based on its chemical alteration history (perms, color treatments, etc.) and its tensile strength, elasticity and level of emolliency, is also evaluated. These have a bearing upon the proportion of repair needed.

In accordance with the invention, the Conditioner Diagnosis also proceeds in four (4) steps distinctive from the selection of a shampoo:
a) Step 1—Consider the Previous Evaluation of Hair Texture.
b) Step 2—Evaluation of Hair Condition: The condition of the hair is evaluated to identify any chemical processes that may be present or still growing out.

Whether the hair is naturally curly or straight is determined. The tensile strength, elasticity and moisture level of hair is checked by doing a stretch test on a single strand of hair. Answers to questions on shine, volume, wet and dry tangling, flyaway and control are obtained.
c) Step 3—Conditioner Selection: The information obtained in Steps 1 and 2 is then used to make a preliminary determination of the requisite level of conditioning for the individual's hair and thus a basic conditioner recommendation in accordance with Table 7 below.

d) Step 4—Reevaluation of Conditioner Selection: Where, upon inspection, the hair is found to be damaged by multiple layers of color, incorrect processing time, or mechanical or environmental factors, it may require additional conditioning as indicated in Table 7.

TABLE 7

| | Current Condition | |
|---|---|---|
| Texture | and/or Damage | Conditioner Needs |
| Fine | Normal/ | Light level conditioner |
| | Single-Processed | Hair color protection |
| | | Address fine texture |
| Fine | Double-Processed/ | Moderate level conditioner |
| | Naturally Curly/ | Hair color protection |
| | Permed/ | Address fine texture |
| | Straightened | |
| Fine | Damaged | Moderately high level conditioner |
| | | Hair color protection |
| | | Address fine texture |
| | | Improve tensile strength and |
| | | repair |
| Medium | Normal/ | Moderate high level conditioner |
| | Single-Processed | Hair color protection |
| | | Address medium texture |
| Medium | Double-Processed/ | Moderately high level conditioner |
| | Naturally Curly | Hair color protection |
| | Permed/ | Address medium texture |
| | Straightened | Improve tensile strength and |
| | | repair |
| Medium | Damaged | High level conditioner |
| | | Hair color protection |
| | | Address medium texture |
| | | Improve tensile strength and |
| | | repair |
| Coarse | Normal/ | Moderately high level of |
| | Single-Processed | conditioner |
| | | Hair color protection |
| | | Address the needs of coarse hair |
| | | Moisturize and tame hair |
| Coarse | Double-Processed/ | Very high level of conditioner |
| | Naturally Curly/ | Hair color protection |
| | Permed/ | Address the needs of coarse hair |
| | Straightened | Moisturize and tame hair |
| Coarse | Damaged | Very high level of conditioner |
| | | Hair color protection |
| | | Address the needs of coarse hair |
| | | Moisturize, repair and tame hair |
| Very | Normal | Very high level of conditioner |
| Coarse | Single-Processed | Hair color protection |
| | | Address the needs of very coarse |
| | | hair |
| | | Moisturize and tame hair |
| Very | Double-Processed/ | Very high level of conditioner |
| Coarse | Naturally Curly/ | Hair color protection |
| | Permed/ | Address the needs of very coarse |
| | Straightened | hair |
| | | Moisturize and tame hair |
| Very | Damaged | Very high level of conditioner |
| Coarse | | Hair color protection |
| | | Address the needs of coarse hair |
| | | Moisturize, repair and tame hair |

### B. The Product Aspect of the Invention

The product aspect of the invention is categorized in at least three (3) parts: 1) proportional shampoos, 2) proportional conditioners and 3) a kit comprising the proportional shampoos and conditioners.

1) The Proportional Shampoos

In accordance with the invention, proportional shampoos are formulated to have specific proportions of cleansing and conditioning properties to address the diagnosed requirements of the hair. Four proportional shampoos have been developed, although, it should be understood, fewer or more proportional shampoos can be developed and used to address the diagnosed requirements of the hair on a more coarse or more fine scale, respectively. As described in further detail below, four proportional shampoos are designated 100/0, 90/10, 80/20 and 60/40. As one moves across the spectrum from 100/0 shampoo to 60/40 shampoo, the level of cleansing activity decreases proportionally while the level of conditioning increases proportionally. A higher level of cleansing activity relative to conditioning corresponds, for example, to a high rate of sebum (oil) production on the scalp and/or to finer, as opposed to coarser hair.

2) The Proportional Conditioners

In accordance with the invention, proportional conditioners are formulated to have specific proportions of conditioning and surfactant ingredients to address the diagnosed requirements of the hair for conditioning, including repair, strengthening and protection. Four proportional conditioners have been developed, although, it should be understood, fewer or more proportional conditioners can be developed and used to address the diagnosed requirements of the hair on a more coarse or more fine scale, respectively. As described in further detail below, four proportional conditioners are designated 5/95, 10/90, 20/80 and 40/60. As one moves across the spectrum from 5/95 conditioner to 40/60 conditioner, the level of conditioning decreases proportionally while the level of surfactant increases proportionally. A higher level of conditioning relative to surfactant corresponds, for example, to any or all of the following diagnoses: a coarse, as opposed to a fine, texture; curly, as opposed to straight, curvature; processed, as opposed to normal, hair; and damage, as opposed to undamaged, hair.

3) The Kit of Proportional Shampoos and Conditioners

The present invention, in addition to being the method which is being described herein, also provides a kit of proportional shampoos and conditioners for practicing the method. The kit includes the proportional shampoos and conditioners described above and diagnostic means to carry out the shampoo and conditioner diagnoses described above to determine which of the proportional shampoos and conditioners is most appropriate for a given person's hair. Examples of the use of the kit will be given below.

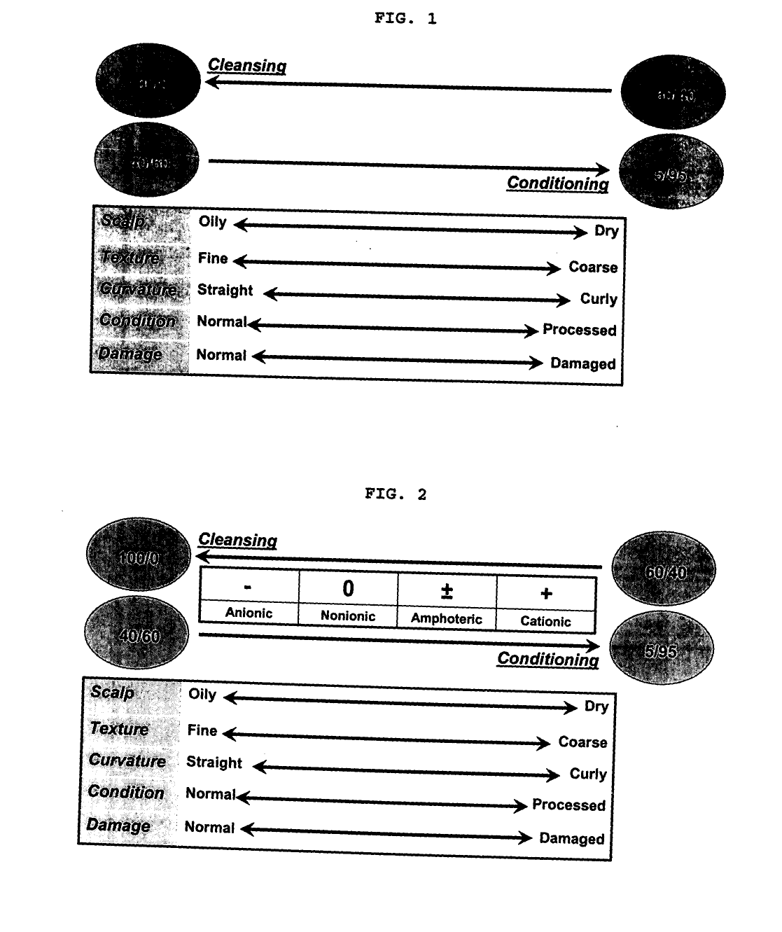

A graphical understanding of the relationship of the method of the invention to the shampoos and conditioners is achieved by reference to FIGS. 1 and 2. FIG. 1 is a diagram exemplifying the proportional relationship of the shampoos and conditioners of the present invention. FIG. 1 places four representative shampoos and conditioners of the invention on a scale indicating relative degrees of cleansing and conditioning as they relate to each of the five (5) criteria for assessing the hair and scalp of an individual. FIG. 2 is the diagram of FIG. 1 modified with an additional scale exemplifying the relationship of formulation components by charge (anionic, neutral, amphoteric, and cationic) as they relate to each of the five (5) criteria for assessing the hair and scalp of an individual.

### C. The Scientific Aspect of Proportional Cleansing and Conditioning

While not wishing to be limited to theory, the advantageous use of the method of the invention is achieved with the proportional cleansing and conditioning formulations, which have been formulated with conventional components known in the art. In accordance with the invention, the proportional shampoos and conditioners are described in terms of a macro system (which refers to the general components in both the shampoo and conditioner formulations) and an ampholyte complex. It will be apparent to those skilled in the art that other shampoo and conditioner formulations can be prepared for use with the method of the invention following the teachings set forth herein. Accordingly, while the shampoo and conditioner formulations are preferred for use with the invention, the method of the invention is not limited to the formulations set forth herein.

1. The Macro System

The macro system refers to the components present in the proportional shampoos and conditioners, and was designed to provide scientific solutions to the full range of indications considered during the diagnostic process that identifies the cleansing and conditioning requirements of an individual's hair. As will be apparent to one skilled in the art, shampoo and conditioner formulations contain a multitude of components to address each and every requisite functionality to be provided by the formulations (e.g., cleansing, conditioning, film formation, strengthening and so on).

While these requirements operate independently, for the purposes of the invention the various components in the shampoo and conditioner are expressed in terms of their relative charges ranging between positive (at one end of the spectrum) and negative (at the other end of the spectrum). As is well known in the art, an individual's hair typically carries a negative charge. Through a correlation of these charges, the macro system provides the benefits of repair, protection, strengthening, and shine enhancing the services of cleansing and conditioning through the establishment of the charge relationships.

An important determinant in both the shampoos and conditioners used in conjunction with the method of the invention is cationic activity provided in part through the use of cationic conditioning agents (or ingredients) in addition to other components of the basic conditioning system. The cationic conditioning agents in the proportional shampoos and conditioners have the capability to repair, protect and strengthen, in addition to providing the basic conditioning services of static removal and wet and dry combability. Overall cationic activity is provided by three generic classes of components interacting within the proportional shampoos and conditioners. The proportional cleansing and conditioning activity of the shampoos and conditioners of the invention is achieved by the proportional (i.e., graduated) concentrations of conditioning agents, surfactants, and ampholytes within each class of components, which are summarized in Table 8 below.

TABLE 8

| Component | Function |
|---|---|
| Surfactant system | Basic cleansing: |
| Anionic (−) | Blend of surfactants |
| Amphoteric (±) | appropriate for |
| Nonionic (0) | specific scalp and hair |
| | cleansing needs |
| Conditioning system | Basic conditioning: |
| Cationic polymers, | Anti Static |
| quaternium ammonium | Wet and dry combability |
| salts (+) | |
| Nonionic (e.g., | |
| silicone, ester) (0) | |
| Ampholyte Complex (±) | Enhanced basic cleansing |
| Amphoteric surfactants | and conditioning: |
| Amino acids | Repair |
| Proteins | Protect |
| | Cleanse |

Each of the components with the above generic classes are well known in the art for use in shampoos and conditioners. Representative examples of compounds in each of these classes are given in any of the following references, which are incorporated herein by reference: “International Cosmetic Ingredient Dictionary and Handbook” (9^{th} Edition, 2002); “Handbook of Cosmetic Science and Technology” (Howard I. Maibach, 2001); “The Science of Hair Care” (Charles Zviac, 1986); “Chemical and Physical Behavior of Human Hair” (Springer-Verlag, 3^{rd} Edition, 1994); “Milady's Standard Textbook of Cosmetology” (2002); and “Hair Structure and Chemistry Simplified” (John Halal, 2002).

A variety of surfactants can be used in accordance with the invention for each class of surfactants. Representative of examples of anionic surfactants to be used include, but are not limited to, anionic surfactants selected from the following classes of surfactants:

1) From about 1 to about 10 percent of an alkyl ether sulfate of the formula (I):
R (OCH₂CH₂)ₙOS₃OX (I)
where “R” is an alkyl group having 12 to 18 carbons, “n” is a integer from 1 to 4, and “X” is a cation such as an alkali metal or a quaternary ammonium group.

2) From about 1 to 10 percent of an alkyl ether sulfosuccinate of the formula (II): 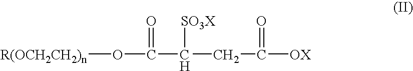
where “R” is an alkyl group having 8 to 20 carbons, “n” is an integer from 1 to 4, and “X” is a cation such as an alkali metal.

Preferably, the anionic surfactant is comprised of sodium laureth sulfate, ammonium laureth sulfate, disodium laureth sulfosuccinate, disodium PEG-12 dimethicone sulfosuccinate or a mixture thereof. Ammonium laureth sulfate and sodium laureth sulfate are available commercially from Cognis Corporation N.A. under the trade names “Standapol EA-1” and “Standapol ES-2”. Disodium laureth sulfosuccinate and disodium PEG-12 dimethicone sulfosuccinate are available commercially from McIntyre Group LTD. under the trade names “Mackanate EL” and “Mackanate DC-50”.

Representative of examples of amphoteric surfactants to be used include, but are not limited to, amphoteric surfactants selected from the following classes of surfactants:

1) From about 1 to 5 percent of an amidoalkyl betaine of the formula (III):
RCONH(CH₂)ₙN⁺R₁R₂CH₂CO₂⁻ (III)
where “RCO” represents a fatty acid derived from a natural oil (e.g., coconut oil), “n” is a single integer from 1 to 3, and R₁ and R₂ are each an alkyl group having 1 to 4 carbons (e.g., methyl groups). A preferred amidoalkyl betaine is cocamidopropyl betaine, which is available commercially from Cognis Corporation N.A. under the trade name “Velvetex BK-35”.

2) From about 0.1 to 5 percent of amphocarboxylate compound having formula (IV):
RCONH(CH₂)ₓN⁺R₁R₂R₃ (IV)
where “R” is an alkyl group having 10 to 21 carbons; “x” is an integer of from about 2 to 4; R₁ is hydrogen; R₂ is a carboxyalkyl group with 2 to 3 carbons; and R₃ is a hydroxyalkyl group containing 2 to 3 carbons. Preferably, the amphocarboxylate compounds are sodium lauroamphoacetate, which is commercially available from McIntyre Group LTD. under the trade name “Mackam HPL 28” and sodium cocobutteramphoacetate, which is supplied by Tri-K Industries under the trade name “Vamasoft Cocoa Butter”.

Representative of examples of nonionic surfactants to be used include, but are not limited to, nonionic surfactants selected from the following classes of surfactants:

1) From about 1 to about 10 percent of long-chain alkyl glucosides or polyglucosides, which are condensation products of (a) long-chain alcohol containing from about 8 to 14 carbon atoms, with glucose or glucose-containing polymer. The alkyl glucosides have about 1 to 6 glucose residues per molecule of alkyl glucoside. A preferred glucoside is lauryl glucoside, which is commercially available from Henkel Corporation under the trade name “Plantaren 1200”.

2) From about 0.1 to about 3 percent of a alkanolamide surfactant having formula (V):
RCONH(CH₂)ₙOH (V)
where RCO represents the fatty acid derived from a natural oil (e.g., coconut oil) and “n” is an integer from 1-3. A preferred alkanolamide is Cocamide MEA, which is available commercially from Protameen Chemicals Inc. under the trade name “Protamide CME”.

3) From about 0.1 to 1 percent of an alkoxylated alcohol, preferably the polyethylene glycol ether of the diester of methyl glucose and oleic acid with an average of 120 moles of ethylene oxides, which is commercially available from Amerchol Corporation under the trade name “Glucamate DOE-120”.

Likewise, cationic conditioning polymers for the conditioning system are well known in the art. For example, representative cationic conditioning polymers include, but are not limited to, cationic conditioning polymers selected from the following:
1) a cationic cellulose derivative; and
2) quaternized copolymers of vinylpyrrolidone and dimethylaminoethyl methacrylate.

The amount of each conditioner component may range, based upon the total weight of the composition, from about 0.01 to 1 percent, preferably from about 0.01 percent to 0.5 percent.

Preferably, the cationic cellulose derivative is a polymeric quaternary ammonium salt derived from the reaction of hydroxyethyl cellulose with a trimethylammonium substituted epoxide. The material known as Polyquaternium-10, supplied by Amerchol Corporation as “Polymer JR-400” having an average molecular weight of 300,000 to 400,000, may be used for this purpose.

A second preferred cationic polymer includes those compounds derived from the reaction of diethyl sulfate and a copolymer of vinyl pyrrolidone and dimethyl aminoethylmethacrylate. The material, known as Polyquaternium-11 and commercially available from ISP under “Gafquat 755N” having an average molecular weight of approximately 1,000,000, may be used for this purpose.

The conditioner system of the invention also includes quaternary ammonium salts. Representative quaternary ammonium salts to be used include, but are not limited to, quaternium ammonium salts selected from the following cationic surfactant classes having formula (VI): 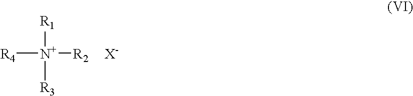
where R₁ is a C₁₄-C₂₂ alkyl group or a C₁-C₄ gluconamidoalkyl group; R₂ and R₃ are each C₁-C₄ alkyl group; R₄ is C₁-C₄ alkyl group or C₁-C₄ hydroxyalkyl group; and X⁻ is a salt-forming anion selected from the group consisting of chloride, bromide, methosulfate and mixtures thereof.

Generally the amount of each of the above conditioning ammonium salts may range based upon the total weight of the composition, from about 0.1 percent to 7 percent, preferably from about 0.1 percent to 5 percent.

One preferred a monoalky quaternary compound is behetrimonium methosulfate, which is commercially available from Croda Inc. under the trade name “Incroquat behenyl TMS” which is a mixture of behetrimonium methosulfate and cetearyl alcohol.

A second preferred conditioning quaternary compound is the gluconamidopropyl dimethyl 2-hydroxyethyl ammonium chloride known as Quaternium-22, which is commercially available from ISP under the trade name “Ceraphyl 60”.

The conditioning system of the invention also includes nonionic silicone-derived compounds (e.g., siloxanes and siloxane derivatives) for use as emollients. Representative silicon-derived compounds to be used include, but are not limited to, non-volatile silicone conditioning agents selected from the following class having formula (VII): 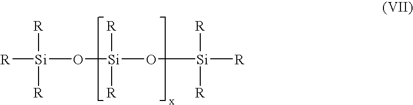
where each “R” independently is alkyl or alkylaryl group, and x is an integer from 7 to 80,000. The silicone is present in an amount of from about 0.1 percent to 10 percent and more preferably from 0.5 percent to 5 percent.

Examples of suitable non-volatile silicone exclusively include polydimethylsiloxane and polymethylphenylsiloxanes having viscosity in the range of 200 to 1000 cps. These materials are known as “Dimethicone 200” available from Dow Corning Corporation and “Si-Tec PTM” series supplied by ISP.

As noted above, the ampholyte system can also include non-surfactant zwitterionic compounds such as amino acids and proteins. Representative zwitterionic compounds to be used include, but are not limited to, an amino-acid-derived zwitterion and a non-hydrolyzed wheat protein in the range of 0.1 to 3 percent by weight.

In a particularly preferred composition, the zwitterions are hydrocreatine commercially available from Centerchem, Inc. under the trade name of Cosmocaire C-100 and non-hydrolized wheat gluten known as “Dragoderm 2/012550” commercially available from Dragoco.

2. The Ampholyte Complex

In accordance with the invention, ampholyte complex refers to the generic class “ampholytes” which are believed to regulate the cationic activity of the shampoos and conditioners by interacting with the other component classes of the macro system. While not wishing to be limited by theory, it is believed that the ampholytes facilitate tailoring of the macro system to specific diagnoses and thus enable optimum cleansing and conditioning. Thus, optimum cleansing and conditioning are achieved by systematically modulating the concentrations of ampholytes that is combined with conditioning agents and surfactants within the broader shampoo and conditioner compositions.

3) The Macro System and Ampholyte Complex for the Shampoos

The macro system for the proportional shampoos uses the interaction of scalp oil production and hair texture to identify ranges in hair type and thus the corresponding cleansing needs for each hair type. As known in the art, texture restricts the transfer of oil (i.e., sebum) from the scalp to the hair establishing the level of conditioning needed, if any, from the shampoo. Likewise, the scalp's oil production determines the level of cleansing needed from the surfactants in the shampoo. The diagnosis of scalp oil production and hair texture establishes the relative proportion of cleansing to conditioning activity needed from a shampoo for a particular hair type.
a) The Surfactant System—Three categories of surfactants are used in the shampoos of the invention represented by negative, neutral and ampholytic charges as exhibited by anionic, nonionic, and amphoteric surfactants, respectively. The surfactants are blended proportionally to address sebum coverage (determined by scalp production) and texture of the hair. The relative concentrations of each class of surfactants are formulated to integrate the activity of the ampholyte in proportion to the hair's demands for emolliency to achieve desired cleansing.
b) The Conditioning System—The functions of static removal and dry and wet combing are addressed in shampooing through the presence of conditioning agents that interact with the ampholytes in the shampoo compositions. The concentration of conditioning agents along with emollients increases in the shampoo compositions based upon the increasing level of conditioning required by an individual's hair as determined from the texture and state of damage relative to the scalp's rate of oil production. In accordance with the invention, it is believed that the intensity of the cationic activity provided by these conditioning agents (e.g., cationic conditioning polymers and quaternium ammonium salts, respectively) increases (i.e., is synergized) by their use in combination with the amphoteric surfactant.
c) The Ampholyte Complex—In the shampoo compositions of the invention, the ampholyte complex is composed primarily of at least one amphoteric surfactant, which, due to the pH of the shampoo composition, is believed to behave as a cationic conditioning agent. As a result, the amphoteric surfactants and the conditioning agents in the shampoo provide the requisite level of conditioning for an individual's hair type. In addition, it is believed that the ampholyte complex (comprised mainly of amphoteric surfactants) also counteracts the anionic surfactant to provide gentler cleansing.

Thus, while not wishing to be limited by theory, it is believed that the general function of the ampholyte complex for the shampoos can be characterized as follows. When applied to the hair, varying proportions of nonionic emollients and amphoteric surfactants influence anionic surfactant activity. The interaction of the amphoteric surfactant in higher concentrations within the shampoo compositions of the invention influences the cleansing activity (by lessening the harsh cleansing characteristics of the anionic surfactants) while synergizing the cationic conditioner agents (by intensifying their basic conditioning, repair and protection performance).

In addition, the shampoos of the invention can contain additional conventional components, such as fragrance, preservatives, anti-oxidants and chelating agents. The preservatives may be any appropriate preservative for a rinse-off product. The chelating agents may be disodium EDTA or trisodium EDTA. The anti-oxidants may be BHT or tocopheryl acetate. One or more of the following fatty alcohols may also be included: cetearyl alcohol, cetyl alcohol and stearyl alcohol. One or more of the following esters may also be included: glyceryl stearate, glycol stearate, triglycerides and meadowfoamates. The humectants glycerin and polyethylene glycols; plant extracts and fragrance may also be included.

4) The Macro System and Ampholyte Complex for the Conditioners

As in the proportional shampoos, the macro system for the proportional conditioners relies on the modulation of cationic components through the systematic use of an ampholyte complex designed to condition, repair, strengthen, protect and enhance the shine of hair in proportion to the needs of a particular hair type.

The macro system for conditioners operates by adjusting the relative cationic activity of the conditioner by altering the ratio of cationic conditioning agents/film formers and the ampholyte complex (which exhibits cationic activity while present in the conditioner) to meet the particular needs of an individual's hair type to provide optimum conditioning, repair, strengthening, shine and protection. The diagnostic determinants that affect the requisite level of cationic activity required by a particular hair type include hair texture, chemical process, curvature, level of damage, and strengthening (in function of the diameter of the substrate).
a) The Surfactant System—In accordance with invention, the surfactant system for the conditioners is composed of an amphoteric surfactant, which is also part of the ampholyte complex described below. While not wishing to be limited by theory, it is believed that the amphoteric surfactant functions by intensifying the activity of the cationic conditioning agents/film formers and other ampholytes in the conditioning composition. The amphoteric surfactant is believed to interact with the other components of the conditioner in two ways:
   (i) The interaction with cationic conditioning polymers—The anti-static and dry/wet combability cationics are intensified by the amphoteric surfactant's own cationic activity; and
   (ii) The interaction with other ampholytes in the ampholyte complex—The amphoteric surfactant intensifies the repairing benefits of the amino acids and proteins.
b) The Conditioning System—As will be apparent to those skilled in the art, a constant level of cationic conditioning polymers is required in all the proportional conditioners to provide a basic level of static removal and improved wet and dry hair combability. While not wishing to be limited by theory, it is believed that the effectiveness of the cationic conditioning polymers and film formers are intensified through their interaction with the ampholytes of the ampholyte complex thereby meeting the increased need for basic conditioning required by higher hair textures.
c) The Ampholyte Complex—The ampholyte complex of the conditioners of the invention include amphoteric surfactants as described above, and at least one additional non-surfactant ampholyte. The additional non-surfactant ampholytes are to provide repair and protection in proportion to the hair's needs and to intensify the basic conditioning provided by the cationic conditioning polymers. Examples of non-surfactant ampholytes to be used include, but are not limited to, zwitterionic compounds such as amino acids and proteins.

Thus, while not wishing to be limited by theory, it is believed that the general function of the ampholyte complex for the conditioners can be characterized as follows. The ampholyte complex (i.e., at least one amphoteric surfactant and at least one non-surfactant ampholyte) in the conditioner is believed to increase adherence of the cationic conditioning polymers and film formers on an individual's hair thereby providing increased improvement to hair quality, both cosmetically and structurally. Preferably, the use of increasing and graduated levels of non-surfactant ampholytes such as amino acids and proteins provide the added properties of repairing and protecting the individual's hair.

As known to those skilled in the art, as texture decreases (i.e., from coarse to fine), the need for deposition with a film former increases (which acts on the relative fragility of the substrate) irrespective of the level of deposition with cationic conditioning agents needed. As a result, an individual's hair will tend to require a conditioner with relatively less or minimal amounts of ampholyte complex such as that provided by the conditioners of the invention designated 20/80 or 40/60.

Likewise, as an individual's hair demonstrates a need for repair (as a result of damage or the presence of chemical hair treatment), the need for repair (through amino acids and proteins) and deposition of cationic conditioning polymers increases. As a result, an individual's hair will tend to require conditioner with relatively greater or maximum amounts of ampholyte complex such as that provided by the conditioners of the invention designated 10/90 or 5/95.

Moreover, hair that lacks surface shine as a result of both texture and damage requires an increased level of cationic conditioning polymer deposition as well as nonionic (silicones) activity.

The following non-limiting examples illustrate the advantageous use of the method of the invention in combination with representative proportional shampoos and conditioners.

### EXAMPLE 1

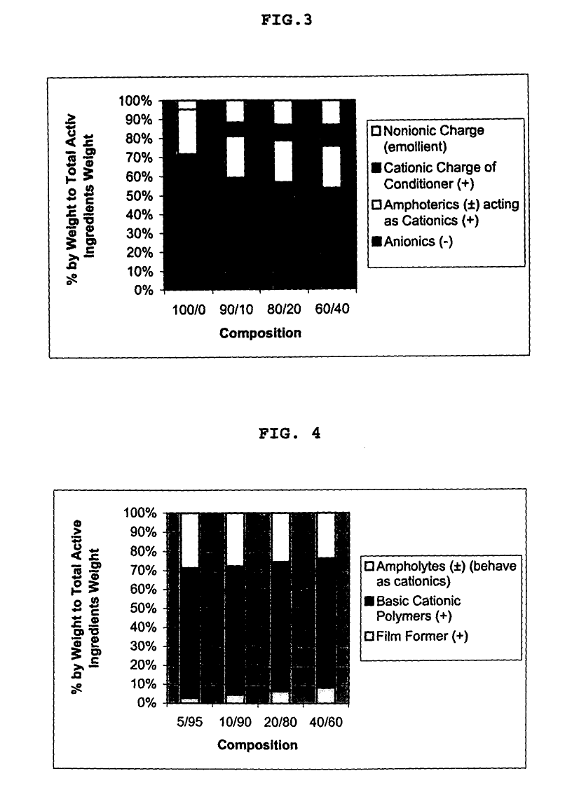

In accordance with the invention, four (4) proportional shampoos were formulated with increasing graduated amounts of ampholyte complex (±charged components), cationic conditioning agents (+charged components) and nonionic emollient/repair agents (0 charged components. The weight percents of the anionic (−charged) components and the other nonionic were kept relatively constant. The shampoos were designated “100/0”, “90/10”, “80/20”, and “60/40” based on the relative cleansing to conditioning activities provided by the components. As will be apparent from the designation “100”, this particular formulation omitted cationic conditioning agents. A listing of the key components (including their charge categories) in the shampoos formulations is set forth in Table 9 below. A bar chart illustrating the general ratio of the components (classified by charge) is also found in FIG. 3. The remaining components included water and common excipients such as fragrance, preservatives, extracts, esential oils among others.

TABLE 9

| | 100/0 | 90/10 | | | |
|---|---|---|---|---|---|
| SHAMPOOS | % | % | 80/20 | 60/40 | |
| % active | WT | WT | % WT | % WT | FUNCTION |
| Meadowfoam | 0.014 | 0.056 | 0.084 | 0.112 | Conditioning |
| amidopropyldimethyl | | | | | |
| betaine | | | | | |
| Cocamidopropyl Betaine | 1.750 | 1.930 | 2.100 | 2.300 | Foam |
| | | | | | booster |
| Sodium | 0.240 | 0.280 | 0.280 | 0.240 | Mild |
| lauroamphoacetate | | | | | cleansing |
| Total % +/− charge | 1.990 | 2.210 | 2.380 | 2.540 | |
| Disodium Peg-12 | 0.360 | 0.360 | 0.360 | 0.360 | Mild |
| dimethicone | | | | | cleansing |
| Sulfosuccinate | | | | | |
| Ammonium Laureth | 3.500 | 3.500 | 3.500 | 3.500 | Mild |
| Sulfate | | | | | cleansing |
| Disodium Laureth | 2.170 | 2.170 | 2.330 | 2.330 | Mild |
| Sulfosuccinate | | | | | cleansing |
| Total % negative charge | 6.030 | 6.030 | 6.190 | 6.190 | |
| Lauryl Glucoside | 6.500 | 6.500 | 6.500 | 6.500 | Mild |
| | | | | | cleansing |
| Cocamide MEA | 1.150 | 1.150 | 1.150 | 1.150 | Foaming |
| | | | | | booster |
| Peg-120 Methyl Glucose | 0.450 | 0.450 | 0.450 | 0.450 | Thickener |
| Dioleate | | | | | |
| Total % nonionic charge | 8.100 | 8.100 | 8.100 | 8.100 | |
| Mixture of Quaternium | 0.000 | 0.840 | 1.010 | 1.390 | Conditioner/ |
| Polymers (including | | | | | anti-static |
| Quaternium-22 and | | | | | |
| Polyquaternium-10) | | | | | |
| Total % positive charge | 0.000 | 0.840 | 1.010 | 1.390 | |
| Dimethicone peg-8 | 0.400 | 1.200 | 1.400 | 1.500 | Emollient/ |
| meadowfoamate | | | | | Repair |

Simplified summaries of the actual weight percents of the categories of components by charge and the weight percentages of the categories relative to each other are listed below in Tables 10 and 11, respectively.

TABLE 10
% of Active Ingredient Net Weight
by Composition - Shampoos

| | 100/0 | 90/10 | 80/20 | 60/40 |
|---|---|---|---|---|
| Anionics (−) | 6.03 | 6.03 | 6.19 | 6.19 |
| Amphoterics (±) acting | 1.99 | 2.21 | 2.38 | 2.54 |
| as Cationics (+) | | | | |
| Cationic Charge of | — | 0.84 | 1.01 | 1.39 |
| Conditioner (+) | | | | |
| Nonionic Charge | 0.40 | 1.20 | 1.40 | 1.50 |
| (emollient) | | | | |
| Total | 8.42 | 10.28 | 10.98 | 11.62 |

TABLE 11
Proportion to Total Active Ingredients
Net Weight - Shampoos

| | 100/0 | 90/10 | 80/20 | 60/40 |
|---|---|---|---|---|
| Anionics (−) | 71.6% | 58.6% | 56.4% | 53.3% |
| Amphoterics (±) acting as | 23.6% | 21.5% | 21.7% | 21.8% |
| Cationics (+) | | | | |
| Cationic Charge of | 0.0% | 8.2% | 9.2% | 12.0% |
| Conditioner (+) | | | | |
| Nonionic Charge (emollient) | 4.8% | 11.7% | 12.7% | 12.9% |
| Total | 100.0% | 100.0% | 100.0% | 100.0% |

### EXAMPLE 2

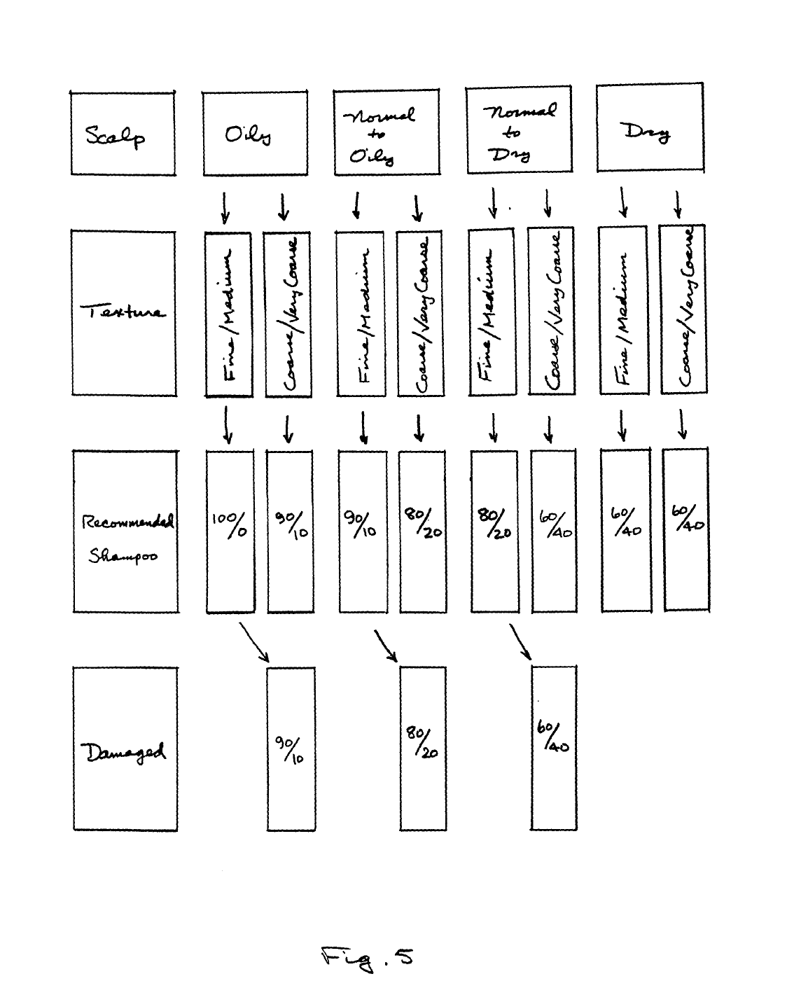

In a manner analogous to Example 1, four (4) proportional conditioners were formulated with decreasing graduated amounts of ampholyte complex (±charged components), and nonionic emollient/repair agents (0 charged components). The weight percentages of some of the cationic conditioning agents (+charged components) were kept relatively constant. All of the formulations omitted anionic (−charged) components since they are used primarily in shampoos. The conditioners were designated “5/95”, “10/90”, “20/80”, and “40/60” based on the relative proportion of surfactant to conditioner. A listing of the key components (including their charge categories) in the conditioner formulations is set forth in Table 12 below. A bar chart illustrating the general ratio of the components (classified by charge) is also found in FIG. 4. The remaining components included water and common excipients such as fragrance, preservatives, extracts, essential oils among others.

TABLE 12

| Conditioners | 5/95 | 10/90 | 20/80 | 40/60 | |
|---|---|---|---|---|---|
| % active | % wt | % wt | % wt | % wt | FUNCTION |
| Behentrimonium | 0.43 | 0.43 | 0.43 | 0.43 | slip/wet |
| Methosulfate | | | | | combing/ |
| | | | | | softening |
| Polyquaternium-37 | 0.65 | 0.65 | 0.65 | 0.65 | Conditioning/ |
| | | | | | Cationic |
| | | | | | thickener |
| Polyquaternium-10 | 0.40 | 0.40 | 0.40 | 0.40 | anti-static |
| Polyquaternium-11 | 0.06 | 0.10 | 0.14 | 0.18 | film former |
| Total % positive | 1.54 | 1.58 | 1.62 | 1.66 | |
| charge | | | | | |
| Phenyl trimethicone | 0.80 | 0.80 | 0.75 | 0.50 | Shine |
| Dimethicone | 3.50 | 3.25 | 3.00 | 2.75 | Shine |
| Total % nonionic | 3.50 | 3.25 | 3.00 | 2.75 | |
| charge | | | | | |
| Sodium | 0.04 | 0.14 | 0.21 | 0.28 | improves |
| cocoabutteramphoacetate | | | | | conditioning |
| Hydrocreatine | 0.50 | 0.40 | 0.30 | 0.20 | repair/increases |
| | | | | | hair tensile |
| | | | | | strength |
| Wheat Gluten | 0.75 | 0.60 | 0.40 | 0.30 | repair/binding to |
| | | | | | damaged hair |
| Total % +/− charge | 1.29 | 1.14 | 0.91 | 0.78 | |

Simplified summaries of the actual weight percents of the categories of components by charge and the weight percentages of the categories relative to each other are listed below in Tables 13 and 14, respectively.

TABLE 13
% of Active Ingredient Net Weight by
Composition - Conditioners

| | 5/95 | 10/90 | 20/80 | 40/60 |
|---|---|---|---|---|
| Film Former (+) | 0.06 | 0.10 | 0.14 | 0.18 |
| Basic Cationic | 1.48 | 1.48 | 1.48 | 1.48 |
| Polymers (+) | | | | |
| Ampholytes (±) (behave | 0.62 | 0.60 | 0.55 | 0.51 |
| as cationics) | | | | |
| Total Combined Cationic | 2.16 | 2.18 | 2.17 | 2.17 |
| Depostion | | | | |

TABLE 14
Proportion to Total Active Ingredients
Net Weight - Conditioners

| | 5/95 | 10/90 | 20/80 | 40/60 |
|---|---|---|---|---|
| Film Former (+) | 2.8% | 4.6% | 6.5% | 8.3% |
| Basic Cationic Polymers (+) | 68.7% | 67.9% | 68.2% | 68.2% |
| Ampholytes (±) (behave as | 28.5% | 27.5% | 25.3% | 23.5% |
| cationics) | | | | |
| Total | 100.0% | 100.0% | 100.0% | 100.0% |

### PROPHETIC EXAMPLE 3

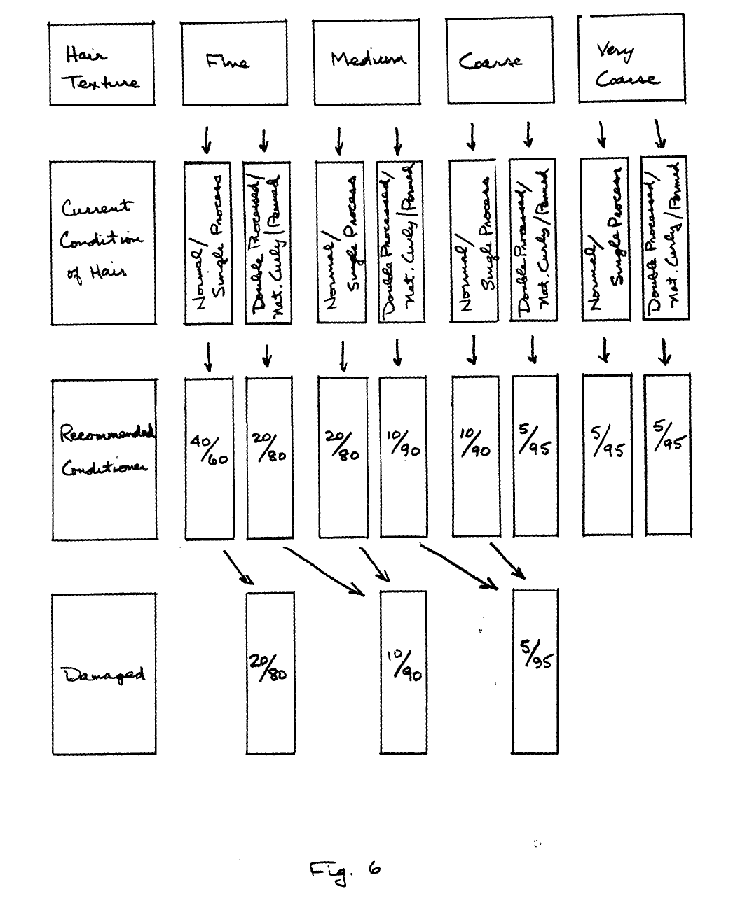

FIG. 5 is a flow chart showing the determination of the proportional shampoo to be used for the eight combinations of scalp type and hair texture. This determination is illustrated below for several possible scenarios.
a) Person shampoos hair daily; if she did not do so, she would notice a significant amount of sebum on the hair and scalp. Scalp type is oily. Hair is seen to be fine, and would clump together and lie flat if not cleansed daily. Referring to FIG. 5, the proportional shampoo recommended is 100/0.
b) Person shampoos hair every other day, if she did not do so, she would notice a significant amount of sebum on the hair and scalp. Scalp type is normal/oily.

Hair is seen to be very coarse, wiry, unruly, curly, and is difficult to control when styling. Referring to FIG. 5, the proportional shampoo recommended is 80/20.
c) Person shampoos daily because of exercise, but scalp is observed to be dry and flaking. Hair has flyaway and is of small diameter, that is, it is fine, and does not readily hold a style. Referring to FIG. 5, the proportional shampoo recommended is 60/40.
d) Person shampoos hair every third day, and likes the way her hair feels after the second day when sebum begins to reach the mid-shaft of her hair. Scalp type is normal/dry. Hair is determined to be of medium texture, but to be damaged following a perm and color treatment. Referring to FIG. 5, the proportional shampoo recommended is 60/40, instead of 80/20, because the hair is damaged.
e) Person shampoos daily; if she did not do so, she would feel as if her scalp was not clean. Scalp type is oily. Hair is seen to be coarse due to large diameter. Referring to FIG. 5, the proportional shampoo recommended is 90/10.

### PROPHETIC EXAMPLE 4

FIG. 6 is a flow chart showing the determination of the proportional conditioner to be used for the eight combinations of hair texture and condition of hair. This determination is illustrated below for several possible scenarios.
a) Hair has previously been determined to be of fine texture, but has highlights that lift its color two levels from its natural color. Condition is considered to be single-processed. Referring to FIG. 6, the proportional conditioner recommended is 40/60.
b) Hair has previously been determined to be coarse, and has not been color-processed, that is, it is normal. Referring to FIG. 6, the proportional conditioner recommended is 10/90.
c) Hair has previously been determined to be of medium texture. Hair has been highlighted with bleach and subsequently colored between the highlights, that is, it has been double-processed. Referring to FIG. 6, the proportional conditioner recommended is 10/90. If hair is considered to be damaged, the proportional conditioner recommended is 5/95.
d) Hair has previously been determined to be coarse, and is naturally curly. Referring to FIG. 6, the proportional conditioner recommended is 5/95.
e) Hair has previously been determined to be of fine texture, and has been permed and is considered to be damaged. Referring to FIG. 6, the proportional conditioner recommended is 10/90, instead of 20/80, because the hair is damaged.

### EXAMPLE 5

Swatches of normal and bleached hair were evaluated for color intensity following a predetermined number of treatments, that is, washing and conditioning, using shampoos and conditioners of the invention. The shampoo designated 100/0 (which contained no conditioning ingredients) was utilized because it would provide the cleanest palette for conditioning analysis.

Two conditioners at opposite ends of the conditioning spectrum 5/95 and 40/60 were selected. According to the diagnostic method of the invention, the 5/95 conditioner would be expected to provide the swatches of bleached hair with optimum conditioning while the conditioner 40/60 would be expected to provide the swatches of normal hair with optimum conditioning.

Six single-processed bleached hair tresses and six treated Piedmont hair tresses where cleaned with a solution of Ammonium Lauryl Sulfate/Propylene glycol (10%/0.35%). 0.35 grams of 100/0 shampoo were applied to each hair tress, massaged for one minute and rinsed under warm water (35° C.) for one minute. Excess water was removed and 0.5 grams of conditioner 5/95 or 40/60 was applied to each tress, massaged into the hair for one minute and rinsed under warm water for one minute. Each tress was then immersed in 0.1% Red 80 Dye solution for one minute and rinsed under warm water (35° C.) for one minute. The tresses were mounted on a tress rack and allowed to oven dry at 40° C. A panel of six people evaluated the tresses under artificial light in a BYK Gardner light box. Tresses were rated on a scale of 1 to 5, 5 being for the most intense red, for color intensity. Tresses were also submitted for color analysis via a Hunter Colorimeter. The results are set forth in Table 15 and graphically depicted in FIG. 7.

TABLE 15

| Bleached Hair After | |
|---|---|
| Ten Treatments | |
| 100/0 and 5/95 | 4.3 |
| 100/0 and 40/60 | 3.9 |
| Difference 5/95 vs. | 0.4 |
| 40/60 | |
| % Difference (of | 10% |
| average) | |
| Normal Hair After Ten | |
| Treatments | |
| 100/0 and 5/95 | 3.5 |
| 100/0 and 40/60 | 3.8 |
| Difference 40/60 vs. | 0.3 |
| 5/95 | |
| % Difference (of | 8% |
| average) | |

As can be seen from Table 15, after ten treatments the swatches exhibited a clear pattern in color intensity depending on conditioner selected. For example, the bleached hair swatches after ten treatments with the 5/95 conditioner averaged a 10 percent improvement in color intensity over its counterparts treated with the 40/60 conditioner. Likewise, the normal hair swatches after ten treatments of the 40/60 conditioner averaged an 8 percent improvement in color intensity over its counterparts treated with the 5/95 conditioner.

### EXAMPLE 6

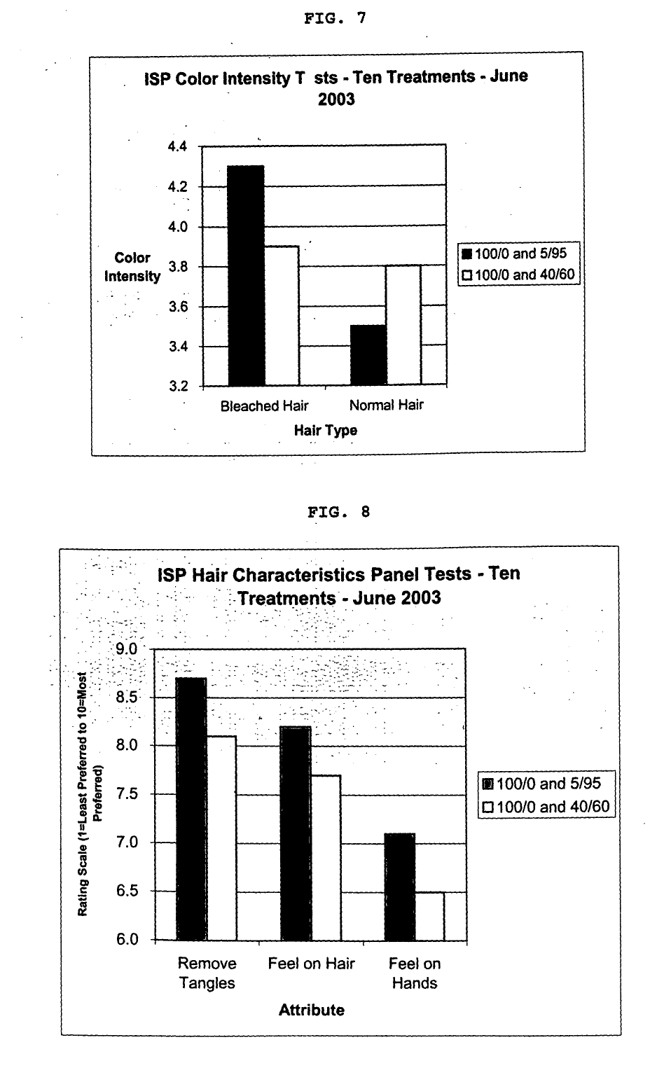

3.5 gram tresses of single-processed bleached hair were treated with 0.35 grams of Shampoo 100/0 (cleansing shampoo). The shampoo was massaged into the hair for 1 minute and rinsed under warm water (35° C.) for 1 minute. 0.5 grams of conditioner (5/95 or 40/60) was applied to each hair tress, massaged into the hair for 1 minute and rinsed under warm water for 1 minute. Wet hair attributes were evaluated remove tangles, feel on hair, feel on hands, by a trained panel (5 people). The results of the evaluations are listed below in Table 16 and graphically depicted in FIG. 8.

TABLE 16
Hair Characteristics Panel Tests

| | | | |
|---|---|---|---|
| Bleached Hair after | Remove | Feel on | Feel on |
| Ten Treatments | Tangles | Hair | Hands |

| | | | |
|---|---|---|---|
| 100/0 and 5/95 | 8.7 | 8.2 | 7.1 |
| 100/0 and 40/60 | 8.1 | 7.7 | 6.5 |
| Difference 5/95 vs. 40/60 | 0.6 | 0.5 | 0.6 |
| % Difference (of average) | 7% | 6% | 9% |

As can be seen from Table 16, swatches conditioned with 5/95 conditioner exhibited a distinct improvement in characteristics over the swatches conditioned with the 40/60 conditioner. While these changes in color intensity and hair characteristics may seem minimal, those skilled in the art will recognize that they provide a highly distinct improvement to an individual's hair. The above color intensity data shows that the diagnostic method of the invention is useful in facilitating optimum cleansing and conditioning of a subject's hair. Likewise, the above hair characteristic data also provides further evidence of the usefulness of the diagnostic method in facilitating optimum cleansing and conditioning of a subject's hair.

### EXAMPLE 7

The effectiveness of the diagnostic method of the invention was evaluated with a Home-Use Test of selected individuals. Thirty-seven (37) individuals, selected at random, were using both professional and mass brands, that is, those purchased from a store, of hair care products. Reference to “professional” hair care products means that the products were purchased in a salon. Likewise, reference to “mass brands” hair care products means that the products were purchased in non-salon environment such as a drugstore. The women range in age from 35-50, were college-educated, and on average earned $40,000+.

Each member of the group was individually diagnosed with the method of the invention for consideration of scalp, texture and condition of hair. They were asked to use the inventive shampoos and conditioners exclusively for a four-week period and to compare the results against their most frequently used shampoos and conditioners.

Each individual was telephoned at the end of the four weeks and asked to evaluate the comparative performance of the inventive shampoos and conditioners that had been diagnosed for their use. Each individual was asked to rate the performance of the inventive shampoos and conditioners to the shampoos and conditioners they most frequently used with the following general criteria: (1) the inventive shampoo and conditioner performed much better; (2) the inventive shampoo and conditioner performed better; (3) the results were the same as I got with my most frequently used products; and (4) the inventive shampoo and conditioner was not as good as my most frequently used products.

In addition, each individual was asked to rate the shampoo in terms of fragrance, texture of product, amount of lathering, experience of lathering, feel of foam, spreadability, ease of rinse, feel of wet hair, detangling, overall shampoo experience, and overall comparison to their most frequently used shampoo. The same evaluation was performed for the conditioners in that each individual was asked to rate the conditioner in terms of fragrance, texture of product, spreadability of conditioner, ease of rinse, feel of wet hair after rinsing out, detangling and combing, overall conditioner experience, and overall comparison to most frequently used product.

Finally, each individual was asked a series of questions addressing the overall state of their hair when dry, which included the smell of hair, the feel of hair, manageability, volume and body, shine, condition of the ends, frizziness, flyaway, softness, combability, effects on hair color, length of time hair stays and feels clean, overall appearance, and overall comparison to their most frequently used products.

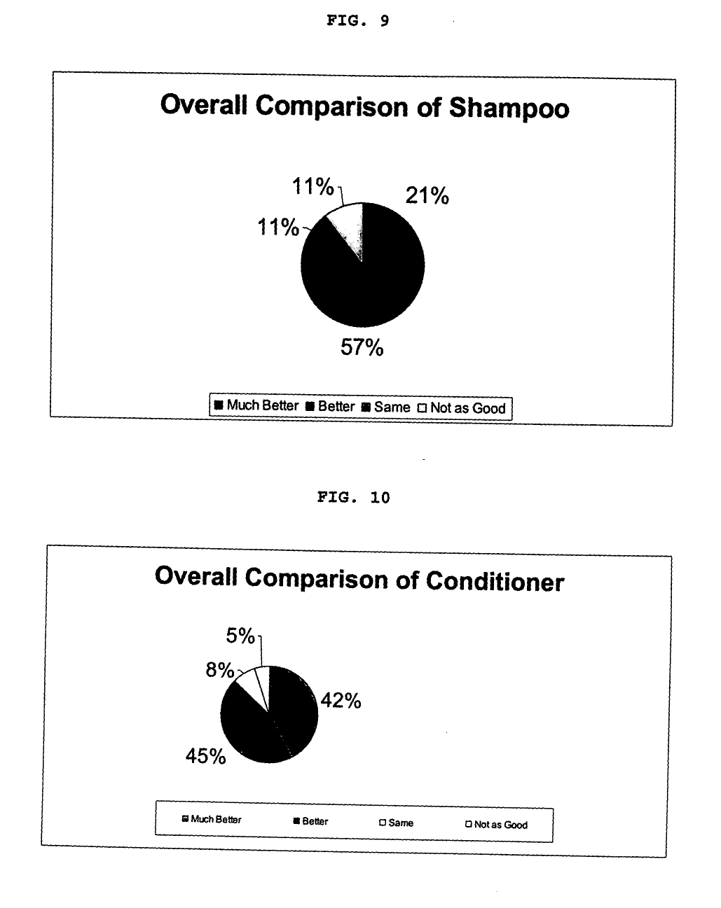

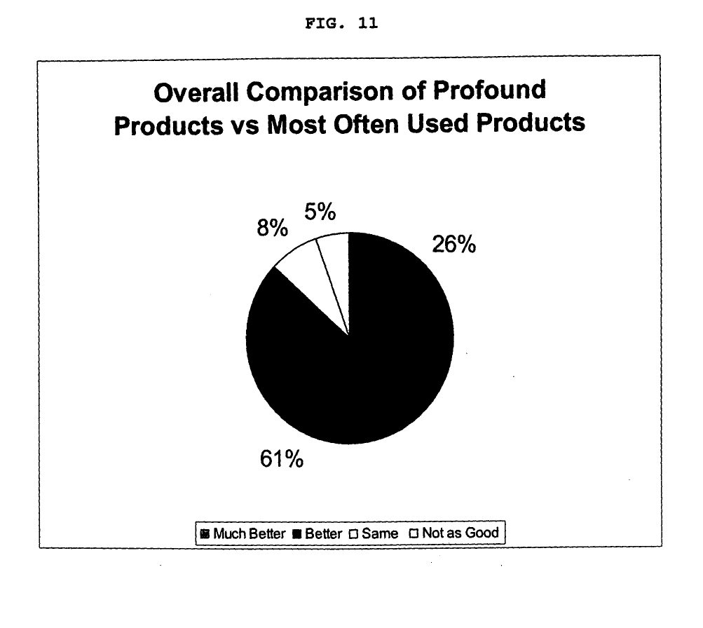

The results of the overall comparisons of the inventive shampoos, conditioners and the combined use of the products are graphically depicted in FIGS. 9 through 11, respectively. As can be seen from FIG. 9, 78 percent of the participants rated their selected shampoo as better or much better than their frequently used shampoo. As can be seen from FIG. 10, 87 percent of the participants rated their selected conditioner as better or much better than their frequently used conditioner. Finally, as can be seen from FIG. 11, 87 percent of the participants rated the overall performance of their shampoo and conditioner as better or much better than their frequently used shampoo and conditioner.

In view of the above, the Home-Use Test study demonstrates that the diagnostic method of the invention enabled shampoos and conditioners to be selected for the majority of the participants that outperformed their most frequently used hair care products.

### Conclusion

The conventional shampoos and conditioners addressing any one market segment, that is, hair type, hair condition, texture, tensile strength, chemical process, or beauty end benefit, typically ignore the considerations of the other segments. The present proportional cleansing and conditioning method, as well as the associated kit of proportional shampoos and conditioners, address the importance of all six segments simultaneously when recommending proportional shampoo and conditioner through the diagnostic aspect that broadens the range of indications considered when formulating shampoos and conditioners, the product aspect designed to align the indications to the individual needs of the consumer, and the scientific aspect which enables the products to perform on the diagnosed substrates.

Modifications to the above would be obvious to those of ordinary skill in the art, but would not bring the invention so modified beyond the scope of the appended claims.

1. A method for diagnosing and appropriately cleansing and conditioning hair comprising the steps of:
a) evaluating a scalp to determine the rate of sebum production thereof;
b) evaluating the hair to determine the texture thereof;
c) recommending a proportional shampoo from a plurality of proportional shampoos, wherein individual proportional shampoos in said plurality include anionic, nonionic and amphoteric surfactants and cationic conditioning polymers, in graduated amounts relative to one another to provide incremental cleansing and conditioning for specific scalp types and hair textures;
d) cleansing the hair and scalp with the recommended proportional shampoo;
e) verifying the texture determined in step b);
f) evaluating the condition of the hair;
g) recommending a proportional conditioner from a plurality of proportional conditioners, wherein individual proportional conditioners in said plurality include at least one amphoteric surfactant, at least one cationic conditioning polymer, at least one film former, and at least one zwitterionic compound selected from the group consisting of amino acids, proteins and combination thereof, in graduated amounts relative to one another to provide incremental conditioning, repair, strengthening, shine and protection for specific textures and hair conditions; and
h) conditioning the hair with the recommended proportional conditioner.

2. A method as claimed in claim 1 wherein said rate of sebum production is expressed in terms of one of a plurality of scalp types.

3. A method as claimed in claim 2 wherein said plurality of scalp types is four scalp types.

4. A method as claimed in claim 3 wherein said four scalp types are identified as oily, normal/oily, normal/dry and dry.

5. A method as claimed in claim 1 wherein the rate of sebum production is determined with reference to the frequency of required washing.

6. A method as claimed in claim 1 wherein the texture is expressed in terms of one of a plurality of texture categories.

7. A method as claimed in claim 6 wherein said plurality of texture categories is four texture categories.

8. A method as claimed in claim 7 wherein said four texture categories are identified as fine, medium, coarse and very coarse.

9. A method as claimed in claim 1 wherein the texture is determined visually and by touch.

10. A method as claimed in claim 1 wherein one of said plurality of said shampoos omits said cationic conditioning polymers.

11. A kit of proportional shampoos and conditioners comprising:
a plurality of proportional shampoos, wherein individual proportional shampoos in said plurality include anionic, nonionic and amphoteric surfactants and cationic conditioning polymers, in graduated amounts relative to one another to provide incremental cleansing and conditioning for specific scalp types and hair textures;
a plurality of proportional conditioners, wherein individual proportional conditioners in said plurality include at least one amphoteric surfactant, at least one cationic conditioning polymer, at least one film former, and at least one zwitterionic compound selected from the group consisting of amino acids, proteins and combination thereof, in graduated amounts relative to one another to provide incremental conditioning, repair, strengthening, shine and protection for specific textures and hair conditions; and
a diagnostic means to identify the appropriate proportional shampoo from said plurality of proportional shampoos and the appropriate proportional conditioner from said plurality of proportional conditioners for the scalp and hair of a particular person.

12. A kit according to claim 11 wherein one of said plurality of shampoos omits said cationic conditioning polymers.

13. A kit according to claim 11 wherein said diagnostic means is a flow chart for identifying said appropriate shampoo and conditioner from said pluralities of proportional shampoos and conditioners.
